(19) United States
(12) Patent Application Publication
(10) Pub. No.: US 2004/0101571 A1
(43) Pub. Date: May 27, 2004

(54) Use of hydrogen peroxide for the treatment of warts

(76) Inventors: Pamela Reed, Knoxville, TN (US); W. Gilmer Reed, Knoxville, TX (US)

Correspondence Address: Pamela Reed, P.O. Box 2309, Knoxville, TN, 37901, US

(21) Appl. No.: 10/647,683

(22) Filed: Aug. 25, 2003

Related U.S. Application Data
(60) Provisional application No. 60/409,854, filed on Sep. 11, 2002.

(51) Int. Cl. A61K 33/40

(52) U.S. Cl. 424/616

(57) ABSTRACT

The present invention provides a method for the treatment of viral-induced tumors, more specifically, human warts. The method uses hydrogen peroxide for the treatment of viral-induced tumors (i.e., warts caused by the human papillomavirus (HPV)) in humans. The method of the invention comprises the topical administration of the hydrogen peroxide or a composition derived there from to the human epidermis and/or to the genital tract as needed. The present invention is also concerned with a unique antiviral composition useful for topical application.

### TECHNICAL FIELD

The present invention generally relates to therapeutic agents for the prevention and treatment of viral-induced tumors, such as warts. In one embodiment, the therapeutic agent is in the form of a gel, comprising hydrogen peroxide and other inactive ingredients. More specifically, the therapeutic agent is peroxide. Use of the gel or its components as a topical agent for the prevention and treatment of viral-induced tumors, such as human papillomavirus-induced tumors, is disclosed.

### BACKGROUND OF THE INVENTION

Viruses which induce tumors in mammals are widespread. Indeed, there are over sixty known types of human papillomaviruses (HPV) which are DNA viruses. These viruses can induce the production of tumors. Some of these HIV's have been associated with benign tumors, such as common warts, while others have been strongly implicated as etiologic agents in dysplasia and carcinomas in the oral and genital mucosa of the infected mammal.

Warts are a very common skin lesion in humans and are caused by various human papillomaviruses (DNA virus). Each virus is related to a specific clinical presentation of the wart. Warts are infectious and can be auto inoculated and spread to other individuals by direct contact.

Verrucae warts have a rough surface, are lumpy and typically flesh colored. Finger-like projections and sometimes dark specks are present, which are the result of thrombosed capillaries. Usually these warts are found on the face and scalp. Plantar warts are found on the planter surface of the feet and can be deep and painful. These warts occur singularly, in clusters or are spread over a wide area Flat warts are typically small, flat-topped, flesh colored papules that occur primarily on the face, hands and forearms. Usually the surface of the wart is smooth and they may appear in the hundreds. Genital warts are soft, flesh colored or slightly pigmented and occur in the genitalia of the mammal and are sexually transmitted. Chronic infections of the viruses that cause genital warts in women are a serious problem as intra epithelial neoplasia or squamous cell carcinoma may develop.

There are various therapies for the treatment of warts, but none are considered truly effective as they typically fail to totally cure the lesions. A discussion of presently accepted therapies can be found in Stone, 1995, Cl. Infec. Diseases, Suppl. 20, pp. 991-997 and Sterling, 1995, Practioner, January 239(1546), pp. 44-47. Numerous compositions are presently marketed for wart removal. One such product is Occlusal.RTM.-HP marketed by the GenDerm Corporation of Lincolnshire, Ill. This product is a 17% solution of salicylic acid in a polyacrylic vehicle. The Shering-Plough Company of Memphis, Tenn. produces and markets a product known as Duo Film.RTM. which is a patch containing salicylic acid. The product literature recommends that the wart be washed and dried prior to the application of a medicated patch which contains 40% salicylic acid. This patch is then covered with an additional bandage and the procedure is repeated every 48 hours until the wart is gone, which sometimes takes up to 12 weeks.

Current modalities for the treatment of viral-induced tumors involve the removal of the tumor by either: (1) surgical intervention (laser or operative); (2) the application of organic acids, such as glacial acetic acid and/or salicylic acid and lactic acid to “burn” the tumor away; (3) the injection into the tumor of an anti-tumor vaccine prepared from ground tumors; and to a lesser extent, (4) the use of a drug, such as podophyllin, interferons and fluorouracil or 5-FU; and (5) freezing.

While being useful for removing the viral-induced tumor, the current treatment modalities still suffer from one or more of the following drawbacks: (1) they can result in the destruction of healthy uninfected tissue; (2) they can result in scarring and disfigurement; (3) they can result in discomfort to the mammal being treated thereby; (4) they can result in necrosis of the tumor and the surrounding tissue may result in a secondary infection which may require treatment with an antibiotic; and (5) they do not always result in the destruction of latent viral DNA which may be maintained in surrounding tissues. Furthermore with these conventional treatments, subjects suffer from significant local, and at times, systemic side effects, incomplete resolution and frequent recurrences of the tumors, and of course, the expense incurred.

There are no references suggesting or disclosing the use of hydrogen peroxide or a gel containing hydrogen peroxide as an agent for the treatment of human papillomavirus-induced tumors. There presently exists in the medical community a need for improved methods and compositions which provide a therapeutic treatment of viral-induced tumors such as warts in humans. The present invention fills that need of the medical community.

### SUMMARY OF THE INVENTION

One aspect of the present invention relates to the use of at least one constituent of hydrogen peroxide or a gel containing hydrogen peroxide for the prevention and treatment of viral-induced tumors. Another aspect of the invention relate to the use of a component or components of said gel to prevent and treat viral-induced tumors in mammals, especially humans. One major benefit of the present invention is that the gel does not destroy healthy, uninfected tissues nor results in significant systemic side effects, local side effects such as irritation, necrosis of tissue surrounding the wart, allergic rashes, scarring, disfigurement or discomfort to the human treated therewith.

Another aspect of the present invention is directed to a simple method for providing therapeutic treatment of viral-induced tumors in humans. An additional aspect of the present invention relates to a method for the destruction of latent viral DNA which is contained in tissues so as to prevent recurrence of these tumors.

Thus, there is disclosed a method for the prevention and treatment of viral induced tumors and skin cancers in a mammal, said method comprising the topical application of hydrogen peroxide.

Also disclosed is a therapeutic composition for the treatment of viral induced tumors in mammals comprising hydrogen peroxide in a pharmaceutically accepted carrier.

There is further disclosed a method for the treatment of genital warts, cancer of the cervix and eradication of human papillomavirus from the female genital tract in infected females, comprising the application of a gel or douche derived from at least one constituent of hydrogen peroxide to the affected area of the human body. There is also disclosed a method for preventing cancer of the cervix, said method comprising the application of hydrogen peroxide to the genital area of a male or female for a period of time and at a sufficient concentration to eradicate the human papillomavirus from the genital area of the male or female.

The method of this invention is specifically directed to the use of a composition that is suitable for topical application. The initial discovery of the inventors was based upon the use of a gel distributed by Proctor & Gamble, known and marketed as “Crest Whitestrips”. The product packaging states that this gel contains the follows: Combine 70% glycerin, 5% carboxypolymethylene, 10% hydrogen peroxide, and 15% water adjusted to pH 6.5 with sodium hydroxide applied to a strip of polyethylene film 0.013 mm thick. The film has an array of shallow pockets, which were typically 0.4 mm across and 0.1 mm deep to accept the gel. The strip of material has a flexural stiffness of about 0.6 grams/cm which followed the surface of the skin overlying the wart. The adhesive surface of the polyethylene film and gel covered the surface of the wart and surrounding skin surface. A second commercially available product, “Plus+White”, was used without the benefit of the polyethylene film. The gel contains water, Poloxamer 407, Glycerin, Hydrogen Peroxide, Sodium Saccharin, Blue 1, Methyl Salicylate, Calcum Disodium EDTA, and Phosphoric Acid. In a further embodiment of this invention, the method of preventing or treating viral-induced tumors uses peroxide that is in a pharmaceutically acceptable carrier such as a gel for topical administration.

There is further disclosed a therapeutic composition for the prevention and treatment of viral-induced tumors in mammals comprising hydrogen peroxide thereof or a derivative thereof in a pharmaceutically acceptable carrier.

In particular, the peroxide itself as the active components of the gel described herein is used for the preparation of therapeutic compositions for the treatment of viral-induced tumors in humans. Preferably, the compositions useful in the method are topically applied to the human in need of such therapy.

The method of the present invention neither destroys healthy, uninfected tissue nor results in any local or systemic side effects, scarring, disfigurement or discomfort to the human treated. Furthermore, the use of the present method results in the destruction of latent viral DNA found in the tumor and the surrounding tissues so that instances of incomplete resolution and are prevented. The method includes the use of the hydrogen peroxide for the administration to an area of the human which presently exhibits viral-induced tumor growth (i.e., warts) eliminating the viral-induced tumor. In accordance with the method according to this invention, “regular use of the hydrogen peroxide” is meant to mean application of the hydrogen peroxide at least once a day to the body surface containing the wart(s). A further embodiment of the method of this invention comprises placing a small amount of gel residue or gel on the tumor to be treated. It has been determined through clinical evaluation that once the method of this invention is initiated, the warts begin to shrink, no matter what size, and will totally disappear after a period of four to six weeks of treatment, or less if the gel is applied more often than once daily at bedtime.

### DESCRIPTION OF PREFERRED EMBODIMENTS

The present invention is based, in part, on the discovery that a commercially available gel manufactured with peroxide is useful for the treatment of viral-induced tumors in humans. More specifically, the invention is directed to the discovery that hydrogen peroxide is the active component of the gel or more specifically the peroxide itself

Wart Destroying Substance

The wart destroying substance is a composition, compound, or mixture capable of influencing or effecting a desired change in appearance and/or structure of the surface it contacts. Examples of appearance and structural changes include, but are not necessarily limited to the shrinking and gradual removal of said wart.

The amount of substance applied to the wart will depend upon the size of the wart. Generally, less than about 1 gram of substance is required. Preferably, from about 0.05 grams to about 0.5 grams and more preferably from about 0.1 gram to about 0.4 grams of the substance is used.

The substance of the present invention can be in the form of a viscous liquid, paste, gel, solution, or other suitable form that can provide sufficient adhesion. Preferably, the substance is in the form of a gel. The substance will have a viscosity of from about 200 to about 1,000,000 at low shear rates. Preferably, the viscosity is from about 100,000 to about 800,000 cps and more preferably from about 400,000 to about 600,000 cps.

Actives suitable for wart destruction include any material safe for on the skin surface which provides wart removal. The actives suitable are selected from the group consisting of the peroxides, metal chlorites, perborates, percarbonates, peroxyacids, and combinations thereof. Suitable peroxide compounds include hydrogen peroxide, calcium peroxide, carbamide peroxide and mixtures thereof. Most preferred is hydrogen peroxide. Suitable metal chlorites include calcium chlorite, barium chlorite, magnesium chlorite, lithium chlorite, sodium chlorite, and potassium chlorite. Additional actives may be hypochlorite and chlorine dioxide. The preferred chlorite is sodium chlorite.

The wart destroying active is present in an amount of from about 0.01% to about 40%, by weight of the substance. If a peroxide compound is chosen as the active, the peroxide compound should provide an amount of hydrogen peroxide equivalent of from about 0.1% to about 20%, preferably from about 0.5% to about 10%, and most preferably from about 1% to about 7%, by weight of the substance. To deliver this amount of hydrogen peroxide equivalent, the peroxide compound, such as hydrogen peroxide, is generally present in an amount of from about 0.1% to about 30% and preferably from about 3% to about 20%, by weight of the substance.

The actives are generally contained in an aqueous gel. The gel is a high viscosity matrix formed from gelling agents known in the art. These gelling agents are safe for topical use, do not readily dissolve on the skin surface, and do not react with or inactivate the compounds incorporated into them. Generally, the gelling agent is a swellable polymer. Furthermore, the gel formed with these agents provides sufficient adhesive attachment to the targeted area of the wart surface. The level of gelling agent to form the gel composition is from about 0.1% to about 15%, preferably from about 1% to about 10%, more preferably from about 2% to about 8%, and most preferably from about 4% to about 7%, by weight of the substance.

Suitable gelling agents useful in the present invention include carboxypolymethylene, carboxymethyl cellulose, carboxypropyl cellulose, poloxamer, carrageenan, Veegum, carboxyvinyl polymers, and natural gums such as gum karaya, xanthan gum, Guar gum, gum arabic, gum tragacanth, and mixtures thereof. The preferable gelling agent for use in the present invention is carboxypolymethylene, obtained from B. F. Goodrich Company under the tradename “Carbopol”. Particularly preferable Carbopols include Carbopol 934, 940, 941, 956 and mixtures thereof. Particularly preferred is Carbopol 956. Carboxypolymethylene is a slightly acidic vinyl polymer with active carboxyl groups. The normal concentration of various carboxypolymethylene resins in water, according to the manufacturer, is below about 2%. However, it has been found that by preparing supersaturated carboxypolymethylene compositions having an absolute concentration in the ranges specified above, suitable high viscosity gel compositions may be prepared.

The concentrated carboxypolymethylene gels have a number of important characteristics in addition to high viscosity. Enough carboxypolymethylene is added to the gel compositions beyond that required to provide high viscosity such that a significant quantity of foot perspiration or water is required to lower the viscosity to the point that the composition may be diluted and washed out by perspiration. The concentrated carboxypolymethylene composition also has a unique tackiness or stickiness which retains and seals against the targeted skin surface it is affixed to, particularly warts.

Water is also present in the gel compositions disclosed herein. The water, employed in the present invention should, preferably, be deionized and free of organic impurities. Water comprises from about 0.1% to 95%, preferably from about 5% to about 90%, and most preferably from about 10% to about 80%, by weight of the substance. This amount of water includes the free water that is added plus that amount that is introduced with other materials.

A pH adjusting agent may also be added to optimize the storage stability of the gel and to make the substance safe for skin surfaces. These pH adjusting agents, or buffers, can be any material which is suitable to adjust the pH of the substance. Suitable materials include sodium bicarbonate, sodium phosphate, sodium hydroxide, ammonium hydroxide, sodium stannate, triethanolamine, citric acid, hydrochloric acid, sodium citrate, and combinations thereof The pH adjusting agents are added in sufficient amounts so as to adjust the pH of the gel composition to about 4.5 to about 11, preferably from about 5 to about 8.5, and more preferably from about 5.5 to about 7. pH adjusting agents are generally present in an amount of from about 0.01% to about 15% and preferably from about 0.05% to about 5%, by weight of the substance.

While the gel described above provides sufficient adhesiveness, additional gelling agents may also be included in the formula to help the active ingredients adhere to the tissues of the skin surface. Suitable agents include both polymers with limited water solubility as well as polymers lacking water solubility. These polymers deposit a thin film on both the wart and surrounding healthy skin surface. Suitable limited water solubility adhesives include: hydroxy ethyl or propyl cellulose. Adhesives lacking water solubility include: ethyl cellulose and polyox resins. Another possible adhesive suitable for use in the instant composition is polyvinylpyrrolidone with a molecular weight of about 50,000 to about 300,000. Still another possible adhesive suitable for use in the instant composition is a combination of Gantrez and the semisynthetic, water-soluble polymer carboxymethyl cellulose.

An additional carrier material may also be added to the substance. Carrier materials can be humectants. Suitable humectants include glycerin, sorbitol, polyethylene glycol, propylene glycol, and other edible polyhydric alcohols. Humectants are generally present in an amount of from about 10% to about 95%, preferably from about 20% to about 80%, and more preferably from about 50% to about 70%, by weight of the substance. In addition to the above materials of the gel of the present invention, a number of other components can also be added to the substance. Additional components include, but are not limited to, xylitol, opacifiers, coloring agents, and chelants such as ethylenediaminetetraacetic acid. These additional ingredients can also be used in place of the compounds disclosed above.

### EXAMPLES

An example of a wart destroying gel is described as follows: Combine 70% glycerin, 5% carboxypolymethylene, 10% hydrogen peroxide, and 15% water adjusted to pH 6.5 with sodium hydroxide. Mix until homogeneous.

Additional examples of an alternative wart destroying gel are described as follows: Combine 56% glycerin, 6% carboxypolymethylene, 10% calcium peroxide, and 24% water.

### Example 1

A seven (7) year old white male presented with warts on the right foot of some two months duration; measuring about 3 mm in diameter, with raised dark spots on the surface. Hydrogen peroxide gel was applied once daily at bedtime over the wart with a Q-tip and left open to air dry. After one week of therapy, the tumor was visibly smaller. After four weeks, the tumor was completely gone and no new tumors were evident.

### Example 2

A second individual, a ten (10) year old female, presented with a 4 mm, raised, wart on her left great toe, began treatment of the wart with hydrogen peroxide gel applied to a surface of a polyethylene film and placed over the wart. After about four weeks of treatment of applying the polyethylene film containing the gel at bedtime the tumor had reduced to a small black dot and was completely gone by the sixth week of treatment applying the gel once daily at bedtime.

### Example 3

A ten (10) year old female presented a large, 3 mm raised and fleshy wart on the dorsum of her left foot. Administration of the carbamide peroxide gel using a Q-tip at bedtime was painted over the wart. After six weeks of treatment, the tumor resolved.

In light of these results, the inventors have concluded that hydrogen peroxide or peroxide itself is an antiviral agent against HPV

It is quite evident from the clinical experience to date, that the hydrogen peroxide of the present invention has been outstandingly effective in the treatment and elimination of warts. The complete eradication of the warts with no recurrence is truly a surprising result as the medical community still searches for a cost effective and efficacious method to control this human malady.

Method of Use

In practicing the present invention, a gel is applied by the user to the surface of a wart. The wart is coated with a gel substance which is preferably in a viscous state to provide not only the active but also tackiness for an extended period of time. The gel readily conforms to the wart surface. The gel is easily removed by the user by washing it off with soap and water. Preferably, each successive treatment will destroy more of the exposed wart surface.

The wart surface is not required to be prepared before the delivery system is applied. For example, the user may or may not choose to wash the skin surface before applying the delivery system. The surface of the wart is not required to be dried or to be excessively wet before the gel is applied.

Preferably, the gel is substantially transparent so as to be almost unnoticeable when worn. Thinness of the delivery system enables the user to walk, move or bend without discomfort.

Preferably, the user applies the delivery system of the present to the wart continuously for about 24 hours a day. Generally, this is done once a day for about 28 to 42 days. The number of days is dependent upon any remaining wart present on the skin surface.

When the user applies more gel on a daily basis, there may be a residue of substance remaining on the surface of the wart. This residual will not be great. If residual substance remains, it may be left in place.

While particular embodiments of the present invention have been illustrated and described, it will be obvious to those skilled in the art that various changes and modifications may be made without departing from the spirit and scope of the invention, and it is intended to cover in the appended claims all such modifications that are within the scope of the invention.

What we claim is:

1. A method for the treatment of human papillomavirus (HPV)-induced tumors in a mammal, said method comprising the topical application of hydrogen peroxide to said mammal.

2. The method according to claim 1 wherein said (HPV)-induced tumor is selected from the group consisting of verrucae warts, plantar warts, flat warts and genital warts

3. The method according to claim 1 wherein said active ingredient is peroxide.

4. The method according to claim 3 wherein said peroxide is in admixture with a pharmaceutically acceptable carrier or excipient.

5. The method according to claim 1 wherein said hydrogen peroxide is in admixture with a pharmaceutically acceptable carrier or excipient.

6. A method for the treatment of genital warts, and treatment of human papillomavirus (HPV) from the female genital tract in infected females comprising the application of a gel comprising hydrogen peroxide.
